(19) United States
(12) Patent Application Publication
(10) Pub. No.: US 2026/0049311 A1
(43) Pub. Date: Feb. 19, 2026

(54) Harnessing MicroRNAs (miRNAs) for Targeted Therapeutics in Cardiac Arrest

(71) Applicant: Mariam Hanna, Fishers, IN (US)

(72) Inventor: Mariam Hanna, Fishers, IN (US)

(21) Appl. No.: 19/232,677

(22) Filed: Jun. 9, 2025

Related U.S. Application Data
(60) Provisional application No. 63/663,240, filed on Jun. 24, 2024.
(60) Provisional application No. 63/668,114, filed on Jul. 5, 2024.
(60) Provisional application No. 63/708,810, filed on Oct. 18, 2024.

(51) Int. Cl. C12N 15/113; A61K 9/00; A61K 31/7105; A61K 33/06; A61K 33/42; A61K 38/57; A61K 47/26

(52) U.S. Cl. CPC C12N 15/113; A61K 9/0019; A61K 31/7105; A61K 33/06; A61K 33/42; A61K 38/57; A61K 47/26; C12N 2310/141

(57) ABSTRACT

The invention pertains to a therapeutic formulation, CARDIOGARDIA, for the management of cardiac arrest. The formulation co-formulates selected microRNAs, including miR-21, miR-1, miR-29, miR-17, and miR-133, and antithrombin and essential electrolytes in a single formulation. The different components in combination target various pathological processes associated with cardiac arrest, including fibrosis, apoptosis, arrhythmia, and coagulopathy. Delivery may be affected by suitable pharmaceutical vehicles or carriers to enable effective delivery. By acting on these distinct pathological processes in combination, the formulation provides a multifaceted approach to stabilizing cardiac function and improving resuscitation outcome. The invention further encompasses methods of treating subjects experiencing cardiac arrest by administering the formulation in therapeutically effective amounts.

### SPECIFIC AIMS

Aim 1: Harnessing MicroRNAs (miRNAs) for Targeted Therapeutics in Cardiac Arrest Rationale: MicroRNAs (miRNAs) are pivotal regulators of gene expression implicated in cardiac arrest pathophysiology. Their ability to modulate critical cellular processes such as inflammation, apoptosis, and fibrosis presents a novel avenue for therapeutic intervention in cardiac emergencies.

### Objectives

1. Identify Dysregulated miRNAs: Utilize advanced sequencing and quantitative PCR techniques to comprehensively profile miRNA expression patterns in cardiac tissues from animal models of cardiac arrest and human post-mortem samples. Focus on miR-21, miR-1, miR-29, miR-17, and miR-133 to elucidate their roles in cardiac remodeling and dysfunction.
2. Functional Validation: Employ bioinformatics analyses and functional assays to validate the regulatory effects of identified miRNAs on key pathways involved in cardiac arrest. Characterize their impact on cardiomyocyte function, apoptosis, and inflammatory responses to prioritize lead candidates for therapeutic development.
   Detailed Extraction Process for miRNAs (miR-21, miR-1, miR-29, miR-17, miR-133)

### 1. Source:

miRNAs can be extracted from tissue samples (e.g., cardiac tissue) or cultured cells.

### 2. Method:

a. Tissue/Cultured Cells Lysis:
Objective: To break open cells and release their contents, including RNA.
Procedure:
   Homogenization: Homogenize the tissue sample or cultured cells in a lysis buffer containing guanidine thiocyanate. This chemical helps to inactivate RNases and denature proteins.
   Lysis Buffer Composition: Typically includes guanidine thiocyanate, β-mercaptoethanol, and Tris-HCl.
      b. Phenol-Chloroform Extraction:
Objective: To separate RNA from DNA, proteins, and other cellular debris.
Procedure:
   Add Phenol-Chloroform: Add an equal volume of phenol-chloroform to the homogenized sample. Vortex the mixture vigorously for 15-30 seconds.
   Centrifugation: Centrifuge at 12,000×g for 15 minutes at 4° C. This separates the mixture into three phases: aqueous (top), interphase (middle), and organic (bottom).
   Aqueous Phase Collection: Carefully transfer the aqueous phase (which contains RNA) to a new tube.
      c. RNA Precipitation:
Objective: To precipitate RNA from the aqueous phase.
Procedure:
   Add Isopropanol: Add 0.5 volumes of isopropanol to the aqueous phase. Mix gently by inverting the tube several times.
   Centrifugation: Centrifuge at 12,000×g for 10 minutes at 4° C. to pellet the RNA.
   Wash Pellet: Wash the RNA pellet with 1 ml of 75% ethanol by gently inverting the tube. Centrifuge at 7,500×g for 5 minutes at 4° C.
   Dry Pellet: Carefully remove the ethanol and air-dry the RNA pellet for 5-10 minutes. Avoid over-drying as it can make the pellet difficult to dissolve.
      d. miRNA Enrichment:
Objective: To enrich miRNAs from the total RNA.
Procedure:
   Commercial miRNA Isolation Kit: Use a kit specifically designed for miRNA isolation (e.g., Qiagen miRNeasy Kit).
   Column-Based Purification: Follow the manufacturer's protocol. Typically, this involves binding RNA to a silica column, washing to remove contaminants, and eluting the enriched miRNAs in a low-salt buffer.
      e. Quality Assessment:
Objective: To assess the quality and quantity of the isolated miRNAs.
Procedure:
   RNA Concentration Measurement: Measure RNA concentration using a spectrophotometer (e.g., NanoDrop). Ideal A260/A280 ratio should be ˜2.0.
   Integrity Check: Assess RNA integrity using agarose gel electrophoresis or an Agilent Bioanalyzer. High-quality RNA will show distinct ribosomal RNA bands without significant degradation.
Aim 2: Integration of Essential Electrolytes for Cardiovascular Stability
Rationale: Electrolyte imbalances exacerbate cardiac dysfunction during and after arrest events. Optimizing electrolyte levels could stabilize cardiac function and support recovery, synergizing with miRNA-based therapies.

### Objectives

1. Characterize Electrolyte Dynamics: Investigate the roles of sodium (Na+), potassium (K+), chloride (Cl—), calcium (Ca2+), magnesium (Mg2+), and phosphate (HPO4\hat{ }2- or H2PO4-) in cardiac electrophysiology and cellular homeostasis during cardiac arrest. Assess their individual and combined effects on cardiomyocyte viability and electrical stability.
2. Formulate Integrated Therapy: Develop a balanced formulation integrating electrolytes with miRNA-based therapeutics to optimize synergistic effects on cardiac function and stability. Utilize nanoparticle delivery systems for targeted and sustained release in cardiac tissues.
Aim 3: Evaluation of Human Plasma-derived Antithrombin in Coagulopathy Management
Rationale: Antithrombin deficiency contributes to thrombotic complications in cardiac arrest. Human plasma-derived antithrombin offers targeted anticoagulant therapy to mitigate clot formation and improve hemostasis.

### Detailed Extraction Process for Human Plasma-Derived Antithrombin

### 1. Source:

Antithrombin is extracted from human plasma, which is obtained from blood donors. The process ensures high purity and activity of the antithrombin protein.

### 2. Method:

a. Plasma Collection:
Objective: To collect human plasma containing antithrombin.
Procedure:
   Donor Screening: Select donors based on health criteria to ensure plasma quality.
   Plasma Collection: Use apheresis to separate plasma from whole blood or collect whole blood and separate plasma by centrifugation.
   Storage: Store the collected plasma at −20° C. or lower until further processing.
      b. Purification:
Objective: To purify antithrombin from the collected plasma.
Procedure:
   Affinity Chromatography:
      Column Preparation: Use heparin affinity columns, which have a high binding affinity for antithrombin.
      Column Equilibration: Equilibrate the column with a suitable buffer (e.g., 20 mM Tris, 150 mM NaCl, pH 7.4).
      Plasma Application: Apply the plasma to the column. Antithrombin binds to the heparin on the column.
      Washing: Wash the column with the equilibration buffer to remove unbound proteins and impurities.
      Elution: Elute antithrombin from the column using a high-salt buffer (e.g., 2 M NaCl) or a gradient of increasing ionic strength.
         c. Concentration and Dialysis:
Objective: To concentrate the purified antithrombin and remove excess salts.
Procedure:
   Concentration:
      Ultrafiltration: Use an ultrafiltration device with an appropriate molecular weight cutoff to concentrate the antithrombin solution.
   Dialysis:
      Buffer Preparation: Prepare a dialysis buffer (e.g., 20 mM Tris, 150 mM NaCl, pH 7.4).
      Dialysis Tubing: Place the concentrated antithrombin solution in dialysis tubing with a suitable molecular weight cutoff.
      Dialysis Process: Dialyze against the buffer at 4° C., with multiple buffer changes to remove excess salts and other small molecules.
         d. Quality Control:
Objective: To ensure the purity, activity, and stability of the purified antithrombin.
Procedure:
   Purity Assessment: Analyze the purity of the antithrombin using SDS-PAGE and densitometry.
   Activity Assay: Measure antithrombin activity using a chromogenic substrate assay or other functional assays.
   Stability Testing: Perform stability tests under various storage conditions to ensure long-term stability.

### Significance

Cardiac arrest remains a leading global cause of mortality, with persistently low survival rates despite advances in emergency medicine. The underlying pathophysiology, characterized by cardiac fibrosis, impaired muscle function, and cellular apoptosis, underscores the critical need for advanced therapeutic interventions beyond current resuscitation techniques. Integrating microRNAs (miRNAs), which regulate gene expression post-transcriptionally, along with essential electrolytes and antithrombin, represents a multifaceted approach to addressing these complex mechanisms.

Incorporation of miRNAs, Antithrombin, and Electrolytes

MicroRNAs (miRNAs), such as miR-21, miR-1, miR-29,miR17, and miR-133, play pivotal roles in mitigating cardiac fibrosis, enhancing muscle contractility, and preventing cell death associated with cardiac arrest. By precisely targeting these molecular pathways, miRNA-based therapies offer a comprehensive strategy to improve cardiac function and patient outcomes. Concurrently, antithrombin, sourced from human plasma to inhibit abnormal clotting, and essential electrolytes (sodium, potassium, chloride, calcium, magnesium, phosphate) stabilize cardiac rhythms, support muscle function, and maintain cellular integrity.

### Storage Conditions for CARDIOGARDIA Formulation

miRNAs (miR-21, miR-1, miR-29, miR-17, miR-133)
Temperature:
   Lyophilized: Store at −20° C. to −80° C.
   Reconstituted: Store at −20° C. for short-term use (up to 1 month) or −80° C. for long-term use (up to 6 months).
Protection From Degradation:
   Aliquoting: Protect from repeated freeze-thaw cycles by aliquoting into small volumes.
Buffer:
   Storage: Store reconstituted miRNAs in a buffer containing RNase inhibitors to prevent degradation.

### Human Plasma-Derived Antithrombin

Temperature:
   Liquid Form: Store at 2-8° C. (refrigerated).
   Lyophilized: For long-term storage, store at −20° C.
Shelf Life:
   Lyophilized: Stable for up to 2 years at −20° C.
   Reconstituted: Use within 24 hours if stored at room temperature or within 1 week if stored at 2-8° C.
Protection From Light:
   Containers: Store in dark or amber-colored containers to protect from light.

### Essential Electrolytes for Cardiovascular Stability (Dextrose and Mannitol)

Temperature:
   Store at room temperature (20-25° C.) or refrigerated (2-8° C.).
Stability:
   Stable for up to 1 year when stored properly.
Protection:
   Store in sealed containers to prevent moisture absorption.

### Injection Preparation and Usage

1. Formulation Preparation:
   Mixing:
      Combine the miRNA solution with the antithrombin solution under sterile conditions.
      Integrate essential electrolytes (dextrose and mannitol) as required.
   Final Volume:
      Adjust the final volume with sterile saline or a suitable buffer.
2. Administration:
   Route:
      Intravenous (IV) or intracardiac injection.

### Mixed Solution Storage

Once the components are mixed to prepare the CARDIOGARDIA injection:
Temperature: Store at 2-8° C.
Duration: Use within 24 hours if stored at room temperature, or within 48 hours if stored at 2-8° C.
Reason for Limited Storage Time: The combined solution may be more prone to degradation and contamination. miRNAs and proteins (antithrombin) can interact, potentially leading to decreased stability and efficacy.

### Chosen Animal Model: Porcine Model (Pigs)

The porcine model is selected for its physiological and anatomical similarities to humans, particularly in cardiac structure and function. This model is ideal for evaluating the efficacy and safety of CARDIOGARDIA in a setting that closely mimics human cardiac arrest.

### Process of the Test

### 1. Study Design

### Objective:

To assess the therapeutic potential of CARDIOGARDIA in improving cardiac function and survival rates post-cardiac arrest.

### Sample Size:

30 pigs divided into three groups (10 pigs per group):
   Group 1: Control group receiving standard treatment (e.g., saline).
   Group 2: Experimental group receiving CARDIOGARDIA.
   Group 3: Placebo group receiving a non-therapeutic formulation.

### 2. Induction of Cardiac Arrest

### Procedure:

Anesthesia: Pigs are anesthetized using a combination of ketamine and xylazine to ensure they are pain-free and immobilized.
Intubation and Ventilation: Animals are intubated and mechanically ventilated to maintain proper respiration.
Cardiac Arrest Induction: Cardiac arrest is induced through either:
   Coronary Artery Occlusion: Occluding a major coronary artery for a specified period to simulate myocardial infarction, followed by reperfusion.
   Electrical Stimulation: Using a defibrillator to induce ventricular fibrillation, mimicking sudden cardiac arrest.

### 3. Administration of Cardiogardia

### Timing:

Administer CARDIOGARDIA immediately after inducing cardiac arrest.

### Dosage:

The dosage is based on preclinical studies, typically ranging from 50-150 IU/kg for antithrombin and 1-10 mg/kg for miRNAs.

### Method:

Intravenous (IV) injection for systemic distribution or direct intracardiac injection if localized delivery is required.

### 4. Post-Administration Monitoring

### Duration:

Monitor pigs for 24-48 hours post-administration for immediate effects and up to 7 days for long-term outcomes.

### Parameters:

Cardiac Function: Use echocardiography and electrocardiogramonitor heart function.
Survival Rate: Record the number of animals surviving each time.
Biomarker Analysis: Collect blood samples to measure cardiac biomarkers (e.g., troponin levels).

### 5. Data Collection and Analysis

### Rounds:

Conduct at least three rounds of the experiment to ensure statistical significance and reproducibility of results.

### Data Points:

Baseline: Measurements before inducing cardiac arrest.
Immediate Post-Administration: Within 1 hour post-CARDIOGARDIA administration.
Short-Term: At 24 and 48 hours post-administration.
Long-Term: Daily monitoring up to 7 days.

### Statistical Analysis:

Compare cardiac function, survival rates, and biomarker levels between groups using ANOVA or similar statistical methods.

### Advancements in Precision Medicine

This research aligns with precision medicine's principles by aiming to personalize treatment strategies based on individual genetic and molecular profiles. Identifying and targeting specific miRNAs dysregulated in cardiac arrest allows for tailored therapeutic interventions. This approach enhances treatment efficacy and minimizes potential side effects by focusing on molecular pathways crucial to cardiac health. Precision in therapy has the potential to revolutionize cardiac arrest management, offering safer and more effective treatment options for patients.

### Bridging the Gap From Bench to Bedside

The translational potential of miRNA-based therapeutics, antithrombin, and electrolytes is substantial. Rigorous preclinical testing in animal models and subsequent clinical trials are essential to validate the safety and efficacy of these interventions. Optimization of delivery systems ensures targeted and sustained effects, crucial for translating laboratory findings into clinical practice. Well-designed clinical trials will evaluate the impact of these combined therapies on human patients, potentially transforming cardiac arrest treatment paradigms and improving patient outcomes significantly.

### Impact on Future Research and Therapeutic Development

Beyond immediate applications in cardiac arrest, insights gained from this research into miRNA regulation, antithrombin function, and electrolyte balance have broader implications for cardiovascular medicine. Discoveries in miRNA roles could lead to new biomarkers for early diagnosis, prognosis, and treatment monitoring across various cardiovascular diseases. Methodologies developed and lessons learned can advance therapeutic strategies for related conditions, expanding the impact of this research in healthcare.

### Enhancing Healthcare Outcomes

Integrating miRNA-based therapies with antithrombin and electrolytes has the potential to transform healthcare outcomes for cardiac arrest patients. By reducing post-arrest complications, enhancing cardiac function, and improving survival rates, these integrated therapies could enhance quality of life and reduce long-term healthcare costs associated with rehabilitation and chronic care. This research contributes to the overarching goal of medical science to improve patient health and well-being through innovative and effective treatment approaches.

### Innovation: Harnessing MicroRNAs for Targeted Therapy in Cardiac Arrest

This research introduces a pioneering approach to using microRNAs (miRNAs) as targeted therapies for cardiac arrest, a condition with significant global mortality rates. Unlike conventional methods focused on immediate resuscitation, this study aims to directly influence molecular pathways implicated in cardiac dysfunction post-arrest.

### Targeted Molecular Intervention

This innovation is central to identifying and precisely targeting specific miRNAs crucial in cardiac pathophysiology. By uncovering dysregulated miRNA profiles associated with cardiac arrest, the research strives to develop interventions that can mitigate cardiac fibrosis, enhance myocardial contractility, and promote cell survival after arrest. This approach shifts treatment paradigms towards personalized, molecularly guided therapies tailored to individual patient profiles.

### Integration of Advanced Technologies

The research integrates cutting-edge technologies in miRNA research and therapeutic development. High-throughput sequencing, bioinformatics analysis, and advanced molecular biology tools are utilized to comprehensively characterize miRNA expression patterns and identify potential therapeutic targets. State-of-the-art methodologies in designing and optimizing miRNA mimics, inhibitors, and delivery systems ensure efficacy, safety, and targeted tissue distribution.

### Translational Research Framework

A hallmark of this research is its strong translational potential, bridging foundational miRNA biology discoveries with clinical applications. Preclinical studies employing rigorous animal models of cardiac arrest provide crucial insights into the efficacy and safety profiles of miRNA-based interventions. Subsequent clinical trials aim to validate these findings in human subjects, aiming for regulatory approval and eventual integration into clinical practice.

### Impact on Cardiovascular Medicine

This innovative use of miRNAs as therapeutic agents holds promise for treating cardiac arrest and extends to broader applications in cardiovascular medicine. Insights gained may lead to developing novel biomarkers for early diagnosis, prognosis, and treatment response assessment across various cardiovascular conditions. By advancing our understanding of miRNA-mediated regulatory networks, this study contributes to the evolution of precision medicine approaches in cardiovascular healthcare.

Approach: Comprehensive Investigation of miRNA Targets in Cardiac Arrest

This research adopts a multifaceted approach to comprehensively investigate miRNA involvement in cardiac arrest, employing advanced molecular biology and bioinformatics techniques.
1. miRNA Profiling: Through high-throughput sequencing and bioinformatics analyses, the study profiles miRNA expression patterns in cardiac tissues from animal models and human samples post-arrest. This rigorous profiling aims to pinpoint miRNAs crucial in cardiac pathophysiology, particularly in regulating fibrosis, apoptosis, and contractility.
2. Selection of Target miRNAs: specific miRNAs are selected for their consistent dysregulation and significant functional implications in cardiac arrest. Notable candidates include miR-21, miR-1, miR-29, miR17, and miR-133, which are recognized for their roles in modulating fibrosis, cardiac muscle function, and stress responses.
3. Functional Validation: Utilizing cardiac cell cultures, in vitro models validate the functional impact of selected miRNAs on relevant cellular processes linked to cardiac arrest. Manipulating miRNA expression levels using mimics or inhibitors elucidates their effects on myocardial integrity and function.

### Integration of Antithrombin and Electrolytes

In addition to miRNA-based therapeutics, the research integrates components targeting antithrombin and electrolytes to enhance therapeutic efficacy in cardiac arrest management.
1. Antithrombin: Incorporating antithrombin into the therapeutic formulation aims to mitigate thrombotic complications associated with cardiac arrest, thereby improving vascular integrity and blood flow regulation crucial for myocardial recovery.
2. Electrolytes: Optimization of electrolyte compositions in the therapeutic regimen supports cardiac function by maintaining optimal cellular ion balance, which is crucial for myocardial contraction and electrical signaling during and post-arrest recovery.

### Development and Optimization of Mirna-Based Therapeutics

Building upon identified targets, the research advances to develop and optimize miRNA-based therapeutics tailored for cardiac arrest intervention.
1. Synthesis of miRNA Mimics and Inhibitors: Synthetic miRNA mimics restore normal miRNA function where downregulated, while antagomirs inhibit overexpressed miRNAs involved in adverse cardiac remodeling. These molecules undergo chemical modifications to enhance stability and specificity.
2. Delivery System Optimization: Advanced delivery systems like lipid nanoparticles and exosomes are employed to achieve efficient and targeted delivery of miRNA therapeutics to cardiac tissues. Optimization focuses on enhancing tissue penetration, cellular uptake, and controlled release kinetics of therapeutic payloads.

### Preclinical Validation and Translational Potential

Critical to the approach is rigorous preclinical validation of miRNA-based therapeutics using relevant animal models of cardiac arrest.
1. Efficacy Studies: Assessment of miRNA mimics and inhibitors in improving cardiac function, reducing fibrosis, and enhancing survival outcomes in animal models undergoing induced cardiac arrest.
2. Safety Evaluation: Comprehensive safety assessments evaluate potential off-target effects, immune responses, and long-term physiological impacts associated with miRNA therapeutic interventions.

### Clinical Translation and Therapeutic Implementation

The research culminates translating preclinical findings to clinical settings, validating the safety and efficacy of miRNA-based therapies in human subjects recovering from cardiac arrest.
1. Clinical Trial Design: Designing phase I/II clinical trials to evaluate pharmacokinetics, pharmacodynamics, and therapeutic outcomes of miRNA-based interventions in cardiac arrest patients.
2. Biomarker Development: Exploration of miRNA biomarkers as diagnostic tools for early detection, prognostication, and treatment response monitoring in cardiovascular diseases, including cardiac arrest.

This integrated approach aims to revolutionize cardiac arrest treatment by leveraging miRNA-based therapeutics alongside antithrombin and electrolytes, offering personalized and targeted interventions to improve patient outcomes.

### SUMMARY OF THE INTERVENTION

The present invention, CARDIOGARDIA, provides a novel therapeutic composition for cardiac arrest therapy. The invention combines microRNA-targeted therapeutics (e.g., miR-21, miR-1, miR-29, miR-17, and miR-133), essential electrolytes, and antithrombin anticoagulant from human plasma into a single composition. This approach addresses four pathological processes of cardiac arrest in a synergistic manner: fibrosis, apoptosis, arrhythmia, and coagulopathy. The combination of optimal delivery vehicles with these ingredients augments cardiac stabilization, survival advantage, and complication prevention of resuscitation.

### BRIEF DESCRIPTION OF THE DRAWINGS

FIG. 1 is a schematic block diagram illustrating the composition of the invention. The figure shows how the ingredients, including selected microRNAs (101), antithrombin (102), and electrolytes (103), are mixed into the formulation herein described as CARDIOGARDIA (104).

FIG. 2 is a diagrammatic representation of the mode of action of the CARDIOGARDIA formulation (201). The figure shows that the use of the formulation results in reduced fibrosis (202), apoptosis (203), arrhythmia (204), and coagulopathy (205).

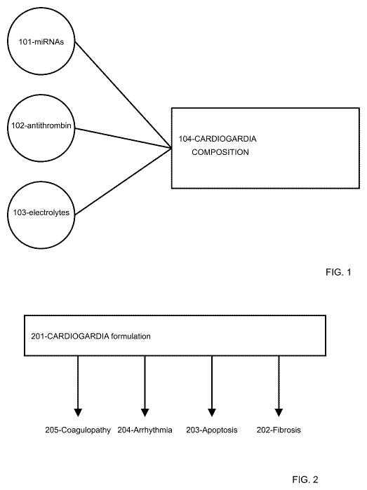

### REFERENCE NUMERALS

101—microRNAs (including miR-21, miR-1, miR-29, miR-17, and miR-133)
102—Antithrombin
103—Electrolytes
104—CARDIOGARDIA composition
201—CARDIOGARDIA formulation
202—Fibrosis (reduced following administration)
203—Apoptosis (reduced following administration)
204—Arrhythmia (reduced following administration)
205—Coagulopathy (reduced following administration)

### Specific Aims

Aim 1: Identification of Dysregulated miRNAs in Cardiac Arrest Objective: To comprehensively profile and characterize miRNA expression patterns in cardiac tissues obtained from animal models and human samples following cardiac arrest. Approach: Utilize high-throughput sequencing and bioinformatics analyses to identify dysregulated miRNAs associated with cardiac dysfunction post-arrest. Validate findings through quantitative PCR and in situ hybridization techniques. Outcome: Identification of key miRNAs implicated in cardiac arrest pathophysiology, providing insights into potential therapeutic targets for intervention.
Aim 2: Functional Validation of Target miRNAs in Cardiac Models Objective: To elucidate the functional roles of selected miRNAs in regulating cardiac fibrosis, apoptosis, and contractility using in vitro cardiac cell models. Approach: Manipulate miRNA expression levels (via mimics and inhibitors) in cardiac cell cultures and assess their impact on cellular processes relevant to cardiac arrest pathology. Utilize functional assays and molecular techniques to validate miRNA-mediated effects. Outcome: Validation of candidate miRNAs as potential therapeutic targets and elucidation of their mechanistic roles in cardiac dysfunction post-arrest.
Aim 3: Development and Optimization of miRNA-Based Therapeutics Objective: To design and optimize miRNA mimics and inhibitors for targeted therapy in cardiac arrest, focusing on efficacy, stability, and delivery. Approach: Synthesize chemically modified miRNA mimics and antagomirs, optimize delivery systems (e.g., lipid nanoparticles, exosomes), and conduct in vitro and preclinical studies to evaluate therapeutic efficacy and safety profiles. Outcome: Development of optimized miRNA-based therapeutics with enhanced stability, efficacy in animal models, and potential for clinical translation.
Aim 4: Preclinical Validation and Safety Assessment Objective: To assess the therapeutic efficacy and safety of miRNA-based interventions in animal models of cardiac arrest.

Approach: Conduct preclinical studies to evaluate the therapeutic effects of miRNA mimics and inhibitors on cardiac function, fibrosis reduction, and survival outcomes. Perform comprehensive safety assessments, including immunogenicity and long-term physiological impacts. Outcome: Demonstrating therapeutic efficacy and safety profiles necessary for advancing miRNA-based therapies to clinical trials.
Aim 5: Clinical Translation and Validation in Human Subjects Objective: To validate miRNA-based therapies'safety, pharmacokinetics, and therapeutic outcomes in patients recovering from cardiac arrest. Approach: Design and conduct phase I/II clinical trials to evaluate miRNA-based interventions in human subjects. Assess pharmacokinetics, pharmacodynamics, biomarker utility, and therapeutic efficacy in patient populations. Outcome: Translation of preclinical findings into clinical practice, with the potential for establishing miRNA-based therapies as novel treatments for improving cardiac recovery and outcomes post-arrest.

### Integration With Public Health and Epidemiological Insights

Cardiac arrest affects over 600,000 people annually in the United States alone, with significant disparities in outcomes based on race, gender, and socioeconomic status. Recent studies underscore the urgency for innovative therapeutic approaches amidst rising mortality rates, particularly highlighted during the COVID-19 pandemic. The development of new medications targeting molecular pathways associated with cardiac arrest, informed by advanced genomics and proteomics, aims to reduce incidence and severity across high-risk populations. Rigorous preclinical and clinical studies will assess safety, efficacy, and regulatory approval, ensuring these novel therapies address critical unmet needs in emergency cardiovascular care.

The Max Planck Institute for Heart and Lung Research (MPI-HLR), located in Bad Nauheim, Germany, is renowned for its pioneering research in cardiovascular and pulmonary biology. The institute's focus on microRNA (miRNA) regulation in cardiac development and disease represents a critical area of investigation within its broader research portfolio.

### Research Focus

### 1. Cardiac Development and Differentiation:

MPI-HLR investigates how miRNAs contribute to the regulation of gene expression during cardiac development and differentiation. MiRNAs play crucial roles in modulating pathways that control the proliferation, differentiation, and maturation of cardiac cells. Understanding these mechanisms is essential for comprehending normal heart development and identifying potential targets for therapeutic interventions in cardiovascular diseases.

### 2. Cardiac Disease Pathogenesis:

Research at MPI-HLR extends to exploring how dysregulation of miRNAs contributes to the pathogenesis of cardiac diseases. By studying miRNA expression profiles in diseased cardiac tissues and animal models, researchers aim to identify specific miRNAs involved in pathological processes such as cardiac hypertrophy, fibrosis, arrhythmias, and heart failure. Insights gained from these studies contribute to the development of new diagnostic markers and therapeutic strategies.

### 3. Experimental Models and Techniques:

The institute employs advanced experimental models and techniques to investigate miRNA function in cardiac biology. This includes genetically engineered animal models, cell culture systems, and high-throughput sequencing technologies to profile miRNA expression and identify miRNA-target interactions. Such approaches are instrumental in elucidating the molecular mechanisms underlying miRNA-mediated regulation in both physiological and pathological contexts.

### Contributions to Science

1. Discovery of Novel miRNA Functions:
MPI-HLR researchers have made significant contributions to discovering novel functions of miRNAs in cardiac biology. The institute enhances our understanding of molecular mechanisms governing cardiac health and disease progression by uncovering specific miRNA-target interactions and their regulatory roles in key signaling pathways.

### 2. Therapeutic Implications:

Insights from MPI-HLR's research on miRNA regulation provide a basis for developing innovative therapeutic approaches. Potential strategies include miRNA-based therapies to restore normal miRNA expression levels or inhibit disease-promoting miRNAs to mitigate cardiac dysfunction and improve patient outcomes.

### 3. International Collaboration and Impact:

The institute collaborates extensively with leading research institutions worldwide, fostering interdisciplinary research collaborations and advancing knowledge exchange in cardiovascular biology and miRNA research. Its findings contribute to global efforts aimed at translating fundamental scientific discoveries into clinical applications for treating cardiovascular diseases.

In conclusion, the Max Planck Institute for Heart and Lung Research stands at the forefront of miRNA research, particularly in the context of cardiac development and disease. Through its innovative approaches and collaborative efforts, MPI-HLR continues to unravel the intricate roles of miRNAs in cardiovascular biology, paving the way for future advancements in therapeutic strategies and precision medicine.

### Medication for Cardiac Arrest (Hypothetical Formulation: CARDIOGARDIA)

1. MicroRNAs (miRNAs):
Description: Small RNA molecules regulating gene expression, potentially modulating pathways in cardiac function and arrest.
Concentration: Ranges from 1×10\hat{ }10 to 5×10\hat{ }10 copies/mL, facilitating targeted therapeutic effects in cardiac tissues.

### 2. Antithrombin:

Description: Derived from human plasma, an anticoagulant protein inhibiting clotting factors during cardiac arrest.
Concentration: Adjusted to ensure compatibility with injection volume (3 mL).

### 3. Electrolytes:

Sodium (Na+): 130-154 mmol/L
Potassium (K+): 3.5-5.0 mmol/L
Chloride (Cl—): 98-106 mmol/L
Calcium (Ca2+): 2.2-2.6 mmol/L
Magnesium (Mg2+): 0.8-1.2 mmol/L
Phosphate (HPO4\hat{ }2- or H2PO4-): 0.8-1.5 Mmol/l
Purpose: Critical for cardiovascular health, maintaining fluid balance, nerve function, and muscle contraction.

### 4. Dextrose:

Concentration: Typically 5% (50 grams per liter), supporting metabolic needs during cardiac recovery.
Amount in 3 mL: Approximately 0.15 grams, providing moderate glucose levels.

### 5. Mannitol:

Description: Osmotic diuretics reduce intracranial and intraocular pressure, with minimal impact on blood sugar compared to dextrose.
Concentration: Selected based on therapeutic goals (commonly 5% to 20%).

### Injection Details:

Total Volume: 3 mL per injection
Purpose: Administered as clinically indicated during or post-cardiac arrest events.

Determining the appropriate dose of CARDIOGARDIA involves a meticulous process aimed at balancing therapeutic efficacy with patient safety. Initially, preclinical studies are crucial, using animal models to explore how the medication behaves in biological systems. These studies help establish a foundation by identifying dose ranges that demonstrate efficacy without causing significant adverse effects. Subsequently, dose-finding studies in humans, typically starting with a low dose in healthy volunteers, provide initial insights into safety and tolerability profiles. As clinical trials progress through phases, data from each stage inform dose adjustments based on pharmacokinetic and pharmacodynamic considerations, patient demographics, and the severity of cardiac arrest. Ethical and regulatory standards guide this process, ensuring that dosing decisions prioritize patient welfare. Continuous monitoring during trials allows for real-time evaluation of efficacy and safety outcomes, enabling researchers and clinicians to refine dosing protocols and optimize treatment benefits for patients.
Research Proposal: Harnessing MicroRNAs (miRNAs) for Targeted Therapeutics in Cardiac Arrest
Aim 1: Identification and Functional Validation of Dysregulated miRNAs in Cardiac Arrest

### 1. Background and Rationale

MicroRNAs (miRNAs) are small, non-coding RNA molecules that play crucial roles in regulating gene expression. miRNAs have been implicated in various pathophysiological processes, including inflammation, apoptosis, and fibrosis. This project aims to identify and validate specific miRNAs as potential therapeutic targets for improving outcomes in cardiac arrest patients.

### 2. Objectives

2.1. Identify Dysregulated miRNAs in Cardiac Arrest Models and Human Samples.
2.2. Validate the Functional Effects of Selected miRNAs on Cardiomyocyte Function and Survival.

### 3. Methods

3.1. miRNA Profiling

Sample Collection: Obtain cardiac tissue samples from animal models of cardiac arrest (e.g., rat or pig models) and human post-mortem samples (in collaboration with local medical examiners). RNA Extraction: Extract total RNA, including miRNAs, using the miRNeasy Mini Kit (Qiagen) $520.00.

miRNA Sequencing: Perform small RNA sequencing using the Illumina NextSeq 500 platform. qRT-PCR Validation: Validate expression levels of miR-21, miR-1, miR-29, miR-17, and miR-133 using TaqMan Advanced miRNA Assays.

### 3.2. Functional Validation

Cell Culture: Maintain H9c2 rat cardiomyoblast cell line and primary human cardiomyocytes. miRNA Transfection: Transfect cells with miRNA mimics or inhibitors (Dharmacon) for miR-21, miR-1, miR-29, miR-17, and miR-133.

Hypoxia-Reoxygenation Model: Subject transfected cells to hypoxia-reoxygenation to mimic cardiac arrest conditions.
Functional Assays:
Cell viability: MTT assay
Apoptosis: Annexin V/PI staining and flow cytometry
Inflammation: qPCR for inflammatory markers (IL-6, TNF-a)
Oxidative stress: Measure ROS levels using DCFDA
Calcium handling: Fura-2 AM calcium imaging

### 4. Expected Outcomes

Identification of differentially expressed miRNAs in cardiac arrest models and human samples.

Validation of the roles of miR-21, miR-1, miR-29, miR-17, and miR-133 in cardiomyocyte function and survival under stress conditions.

Potential identification of novel miRNA targets for therapeutic intervention in cardiac arrest.

### 5. Timeline

Months 1-3: Sample collection and miRNA profiling
Months 4-6: qRT-PCR validation and initial functional assays
Months 7-9: Complete functional validation experiments
Months 10-12: Data analysis and manuscript preparation

### 6. Budget Estimation

Obtaining miRNA Molecules:
order synthetic miRNA mimics and inhibitors from companies specializing in RNA oligonucleotides. Some reputable suppliers include:
Dharmacon (Horizon Discovery)
Qiagen
Ambion (Thermo Fisher Scientific)
Sigma-Aldrich

The miRNAs of interest (miR-21, miR-1, miR-29, miR-17, and miR-133), will need to order both mimics and inhibitors to study their effects when overexpressed or inhibited.

Evaluating miRNA Activities in Cell Lines:

I can use the H9c2 rat cardiomyoblast cell line or primary human cardiomyocytes for the experiments. Here's a basic workflow:
a. Culture cells in appropriate media
b. Transfect cells with miRNA mimics or inhibitors using lipid-based transfection reagents
c. Subject cells to hypoxia-reoxygenation to mimic cardiac arrest conditions
d. Perform functional assays as outlined in the proposal (cell viability, apoptosis, inflammation, oxidative stress, and calcium handling)

### Cost Estimation:

Here's a rough estimate of the costs for the main components of the experiments:
miRNA mimics and inhibitors: $200-$300 per miRNA (×5 miRNAs)=$1,000-$1,500
Cell culture media and supplements: $500-$1,000
Transfection reagents: $200-$300
RNA extraction kits: $300-$500
qRT-PCR reagents and primers: $1,000-$1,500
Functional assay kits (MTT, Annexin V, DCFDA, etc.): $1,500-$2,000
Misc. lab supplies (pipette tips, tubes, etc.): $500-$1,000
Total estimated cost: $5,000-$7,800
   Sample Collection and miRNA Profiling Protocol for Human Post-Mortem Samples

### 1. Sample Collection

### 1.1. Ethical Considerations:

Ensure proper ethical approval and consent are obtained
Follow all relevant regulations for handling human post-mortem tissue

### 1.2. Post-Mortem Interval (PMI):

Record the time between death and sample collection
Aim to minimize PMI as RNA degradation increases with time

### 1.3. Tissue Collection:

Collect tissue samples as soon as possible after death
Use sterile, RNase-free tools to avoid contamination
Cut tissue into small pieces (approximately 0.5 cm³)
Immediately snap-freeze samples in liquid nitrogen
Store at −80° C. until RNA extraction

### 1.4. Documentation:

Record relevant metadata (age, sex, cause of death, medical history)
Note any conditions that might affect RNA quality (e.g., agonal state, fever)

### 2. RNA Extraction

2.1. Choose an RNA extraction method optimized for degraded samples:
Consider kits designed for FFPE (formalin-fixed paraffin-embedded) tissues
Methods like TRIzol followed by column purification can be effective

### 2.2. Process Samples in an RNase-Free Environment:

Use dedicated equipment and reagents
Clean work surfaces with RNase decontamination solution

### 2.3. Homogenize Tissue Thoroughly:

Use mechanical disruption (e.g., bead-beating) or chemical lysis
Ensure complete homogenization for optimal RNA yield

### 2.4. Assess RNA Quality and Quantity:

Use spectrophotometry (e.g., NanoDrop) for concentration and purity
Use Bioanalyzer or similar for RNA integrity number (RIN)
Expect lower RIN values compared to fresh samples
   3. miRNA Profiling

### 3.1. Select a Profiling Method Suitable for Potentially Degraded Samples:

qRT-PCR arrays are often preferred due to their sensitivity
NGS can provide comprehensive profiling if RNA quality is sufficient
   3.2. For qRT-PCR Arrays:
Use protocols optimized for low-input or degraded RNA
Consider pre-amplification steps if RNA quantity is limited

### 3.3. For NGS:

Use library preparation kits designed for low-quality or FFPE samples
Adjust fragment size selection to account for RNA degradation

### 4. Data Analysis

### 4.1. Account for Post-Mortem Factors in the Analysis:

Consider PMI as a covariate in statistical models
Analyze the impact of RNA quality metrics on results

### 4.2. Use Appropriate Normalization Strategies:

Consider using multiple reference genes or global mean normalization
Evaluate the stability of commonly used reference miRNAs in the samples

### 4.3. Validate Findings:

Use individual qRT-PCR assays to confirm key results
Consider validating in an independent cohort if possible

### 5. Functional Analysis and Interpretation

5.1. Consider the Impact of Post-Mortem Changes on miRNA Expression 5.2. Interpret Results in the Context of Ante-Mortem Conditions and Post-Mortem Changes 5.3. Correlate miRNA Changes With Histological or Biochemical Markers if Available
Timeline for Sample Collection and miRNA Profiling

### 1. Sample Collection: 1-2 Hours Per Sample

Tissue collection: 15-30 minutes
Processing and freezing: 30-60 minutes
Documentation: 15-30 minutes

### 2. RNA Extraction: 3-4 Hours

Sample preparation: 30 minutes
Extraction procedure: 2-3 hours
Quality control: 30 minutes
   3. miRNA Profiling: 1-7 Days
   qRT-PCR Arrays:
cDNA synthesis: 2-3 hours
qPCR setup: 1-2 hours
qPCR run: 2-4 hours
Data collection: 1-2 hours Total: 1-2 days

### Microarray:

Sample labeling: 1-2 days
Hybridization: 12-16 hours
Washing and scanning: 2-3 hours
Data extraction: 1-2 hours Total: 2-3 days

### Next-Generation Sequencing (NGS):

Library preparation: 1-2 days
Sequencing: 1-3 days (depending on the platform)
Initial data processing: 1-2 days Total: 3-7 days

### 4. Data Analysis: 3-14 Days

Initial analysis and normalization: 1-3 days
Statistical analysis: 1-3 days
Functional analysis and interpretation: 1-7 days

### 5. Validation: 1-7 Days

qRT-PCR for selected miRNAs: 1-2 days
Data analysis and integration: 1-5 days
   Total time From Sample Collection to Final Analysis:
Minimum (using qPCR arrays): ˜1-2 weeks
Maximum (using NGS with extensive analysis): ˜4-6 weeks

Note: These timelines are approximate and can vary based on sample number, equipment availability, experience level, and complexity of the analysis.

Timeline for qRT-PCR Validation and Initial Functional Assays
1. qRT-PCR Validation: 3-5 days

### Day 1: Experimental Design and Primer Optimization (4-6 Hours)

Select miRNAs for validation
Design or order primers
Optimize PCR conditions if using new primers
   Day 2: cDNA Synthesis and qPCR Setup (4-5 Hours)
Reverse transcription of RNA to cDNA: 2-3 hours
Prepare qPCR reactions: 1-2 hours
   Day 3: qPCR Run and Initial Data Analysis (6-8 Hours)
Run qPCR: 2-4 hours (depending on the number of samples and targets)
Initial data analysis: 2-4 hours

### Day 4-5: Data Analysis and Interpretation (8-16 Hours)

Comprehensive data analysis: 4-8 hours
Statistical analysis: 2-4 hours
Result interpretation and comparison with initial profiling data: 2-4 hours

### 2. Initial Functional Assays: 1-3 Weeks

### Week 1: Assay Selection and Preparation (3-5 Days)

Choose appropriate functional assays based on miRNA targets: 1 day
Prepare or order necessary reagents: 1-2 days
Set up cell cultures or prepare samples: 1-2 days

### Week 2: Conduct Functional Assays (3-5 Days)

Perform assays (e.g., luciferase reporter assay, Western blot): 2-3 days
Data collection: 1-2 days

### Week 3: Data Analysis and Interpretation (2-3 Days)

Analyze assay results: 1-2 days
Integrate with qRT-PCR validation data: 1 day

Total time for qRT-PCR validation and initial functional assays: 2-4 weeks

Timeline for Complete Functional Validation Experiments

### 1. Experimental Planning and Preparation: 1-2 Weeks

Literature review and hypothesis formulation: 2-3 days
Experimental design: 2-3 days
Ordering necessary reagents and materials: 1 week (can overlap with other tasks)

### 2. In Vitro Studies: 4-8 Weeks

Cell Culture and Transfection: 2-3 weeks
Cell line selection and cultivation: 1 week
Optimization of transfection conditions: 3-5 days
Transfection experiments (miRNA mimics/inhibitors): 1 week
Molecular Assays: 2-3 weeks
qRT-PCR for target gene expression: 2-3 days
Western blot for protein level changes: 3-5 days
Luciferase reporter assays: 1 week
Functional Assays: 2-3 weeks
Cell proliferation assays: 3-5 days
Apoptosis assays: 2-3 days
Migration/invasion assays: 1 week
Other specific assays based on miRNA function: 1-2 weeks

### 3. In Vivo Studies (If Applicable): 8-16 Weeks

Animal model selection and acquisition: 2-4 weeks
Acclimation period: 1 week
Treatment period (e.g., miRNA delivery): 4-8 weeks
Sample collection and processing: 1 week
Data analysis from in vivo experiments: 2-3 weeks

### 4. Advanced Molecular Analyses: 3-6 Weeks

RNA-seq or proteomics experiments: 2-3 weeks
Bioinformatics analysis: 1-3 weeks

### 5. Data Analysis and Integration: 2-4 Weeks

Statistical analysis of all experiments: 1-2 weeks
Integration of in vitro and in vivo data: 1-2 weeks

### 6. Validation of Key Findings: 2-4 Weeks

Repeat critical experiments: 1-2 weeks
Additional assays to address reviewers'potential concerns: 1-2 weeks

### 7. Manuscript Preparation: 4-8 Weeks

Writing and Editing: 3-6 Weeks
Figure preparation: 1-2 weeks
Total time for complete functional validation: 6-12 months

Note: This timeline can vary significantly based on the complexity of the miRNA function, the number of target genes, the availability of resources, and any unexpected results that may require additional experiments.

Timeline for Data Analysis and Manuscript Preparation in miRNA Studies

### 1. Data Analysis: 4-8 Weeks

### Week 1-2: Initial Data Processing and Quality Control

Organize and compile all experimental data: 2-3 days
Perform quality control checks: 1-2 days
Normalize data across experiments: 2-3 days

### Week 3-4: Statistical Analysis

Perform statistical tests appropriate for each experiment: 3-5 days
Create visualizations (graphs, charts) of key results: 2-3 days
Identify significant findings and trends: 2-3 days

### Week 5-6: Integration and Advanced Analysis

Integrate results from different experiments: 3-5 days
Perform pathway analysis or other bioinformatics analyses: 3-5 days
Consult with a statistician or bioinformatician (if needed): 1-2 days

### Week 7-8: Final Analysis and Interpretation

Refine analyses based on integrated results: 3-4 days
Interpret results in the context of existing literature: 3-4 days
Identify key conclusions and novel findings: 2-3 days

### 2. Manuscript Preparation: 6-10 Weeks

### Week 1-2: Outline and Introduction

Create a detailed outline of the manuscript: 2-3 days
Write introduction, including literature review: 5-7 days

### Week 3-4: Methods and Results

Write detailed methods section: 3-5 days
Describe results, referring to figures and tables: 5-7 days

### Week 5-6: Discussion and Abstract

Write discussion, interpreting results and comparing to literature: 7-10 days
Compose abstract summarizing key findings: 1-2 days

### Week 7-8: Figures, and Tables

### Week 9-10: Review and Revision

Internal review: 5-7 days
Revise based on feedback: 3-5 days
Final proofreading and formatting: 2-3 days

### Total Time for Data Analysis and Manuscript Preparation: 10-18 Weeks

Note: This timeline can vary based on the complexity of the study, the number of experiments, the experience of the team, and other ongoing commitments. Collaboration and parallel work on different sections can potentially shorten the overall time.

Comparison: Purchasing vs. Extracting microRNA
Purchasing microRNA

### Advantages:

1. Time-saving: Eliminates the need for sample collection and RNA extraction steps
2. Consistency: Commercially available microRNA often has standardized quality and concentration
3. Availability: Can obtain microRNAs that might be difficult to extract from biological samples
4. Reduced variability: Minimizes issues related to sample quality and extraction efficiency
5. Immediate use: Ready for experimental use upon arrival

### Disadvantages:

1. Cost: Can be more expensive, especially for large-scale studies
2. Limited context: Lacks the biological context of the original sample
3. Potential differences: May not perfectly represent the microRNA profile in the specific biological context
   Extracting microRNA

### Advantages:

1. Biological relevance: Directly represents the microRNA profile in the specific samples
2. Cost-effective for large-scale studies: Can be cheaper when processing many samples
3. Flexibility: Allows for extraction of all microRNAs present in the sample
4. Context preservation: Maintains the relationship with other molecular factors in the sample

### Disadvantages:

1. Time-consuming: Requires sample collection and RNA extraction steps
2. Variable quality: RNA quality can vary based on sample handling and extraction efficiency
3. Technical expertise: Requires skill and experience for consistent, high-quality extractions
4. Potential for contamination: Risk of introducing contaminants during the extraction process
   Process for Integrating miRNA Findings with Animal Model of Cardiac Arrest
1. Selection of Candidate miRNAs; select 2-3 top candidate miRNAs that show the most significant dysregulation and functional effects related to cardiac arrest.
2. Animal Model Selection; Choose an appropriate animal model of cardiac arrest. Common models include:
Ventricular fibrillation (VF) induced cardiac arrest in pigs or dogs
Asphyxia-induced cardiac arrest in rats or mice
Potassium chloride (KCl) induced cardiac arrest in rodents

Consider the strengths and limitations of each model concerning the specific miRNA targets and hypotheses.

3. Baseline miRNA Expression Analysis

Before inducing cardiac arrest, collect cardiac tissue samples from healthy animals.

Analyze the expression of the candidate miRNAs in these samples to establish a baseline.

### 4. Cardiac Arrest Induction and Resuscitation

Induce cardiac arrest using the chosen method.

Perform cardiopulmonary resuscitation (CPR) and defibrillation as per standard protocols.

Collect cardiac tissue samples at various time points post-resuscitation (e.g., 1 hour, 6 hours, 24 hours).

5. miRNA Expression Analysis in Cardiac Arrest Model

Analyze the expression of the candidate miRNAs in the collected cardiac tissue samples.

Compare the expression levels to the baseline and look for changes correlating in vitro findings.

### 6. Functional Assessments

Perform relevant functional assessments on the animals, such as:
Echocardiography for cardiac function
Electrocardiogramarrhythmias
Neurological deficit scores for post-cardiac arrest brain injury
Correlate these functional outcomes with miRNA expression changes.

### 7. Histological and Molecular Analyses

Conduct histological examinations of the heart tissue for signs of damage or remodeling.

Perform molecular analyses (e.g., qPCR, Western blot) to assess the expression of predicted target genes and proteins of the miRNAs.

8. miRNA Modulation In Vivo

For upregulated miRNAs: Use antagomirs or locked nucleic acid (LNA) inhibitors

For downregulated miRNAs: Use miRNA mimics or adeno-associated virus (AAV) vectors for overexpression

Administer these modulators before inducing cardiac arrest and assess their effects on cardiac arrest outcomes.

### 9. Mechanistic Studies

Investigate the mechanisms by which miRNAs affect cardiac arrest outcomes.

This may involve analyzing specific pathways (e.g., apoptosis, calcium handling, oxidative stress) in the animal model tissues.

### 10. Therapeutic Potential Assessment

If miRNA modulation shows beneficial effects, conduct additional experiments to assess:
Dose-response relationships
Timing of administration (pre-arrest, during CPR, post-resuscitation)
Potential off-target effects in other organs
Aim 1: Harnessing MicroRNAs (miRNAs) for Targeted Therapeutics in Cardiac Arrest Mechanism of miRNA Therapeutics:

MicroRNAs are small, non-coding RNAs that regulate gene expression post-transcriptionally by binding to messenger RNA (mRNA) and either blocking its translation or marking it for degradation. For cardiac arrest, miRNAs such as miR-21, miR-1, miR-29, miR-17, and miR-133 are known to influence vital cellular pathways that contribute to cardiac damage, including: miR-21: Known for promoting fibrosis and cardiac remodeling, miR-21 is associated with increased inflammation and scarring in the heart after ischemic injury. Targeting this miRNA could help reduce pathological remodeling.
miR-1: It plays a role in cardiomyocyte electrical activity. Dysregulation of miR-1 is linked to arrhythmias, and normalizing its levels could restore electrical stability.
miR-29: Involved in regulating extracellular matrix proteins, crucial in preventing excessive fibrosis and stiffening of the heart muscle. Inhibiting miR-29 could prevent deleterious tissue remodeling.
miR-133: Critical for maintaining cardiomyocyte survival and function, miR-133 has anti-apoptotic effects. Enhancing its levels could prevent cell death, preserving cardiac muscle function after an ischemic event.

By modulating the expression of these miRNAs, the aim is to prevent inflammation, fibrosis, and apoptosis while improving heart muscle function post-cardiac arrest. This could improve survival rates and recovery.

### Technical Process:

The proposed miRNA extraction process involves RNA isolation techniques using tissue samples and cultured cells. After lysis and separation, miRNAs are enriched, typically using commercial isolation kits (e.g., Qiagen's miRNeasy kit). The next step involves functional validation, where bioinformatics and in vitro assays assess the impact of miRNAs on key pathways like apoptosis, fibrosis, and cardiomyocyte viability.
Aim 2: Integration of Essential Electrolytes for Cardiovascular Stability Mechanism of Electrolytes:
Electrolytes such as sodium (Na+), potassium (K+), calcium (Ca2+), magnesium (Mg2+), and phosphate play essential roles in cardiac electrophysiology and cellular homeostasis:
Sodium and Potassium: These ions regulate action potentials and membrane depolarization.
Potassium, in particular, maintains resting membrane potential and helps in repolarization after a cardiac action potential.
Calcium: Vital for excitation-contraction coupling in cardiomyocytes. Calcium influx through voltage-gated calcium channels triggers muscle contraction, while its efflux helps in relaxation.
Magnesium: Acts as a cofactor for many enzymes, including those involved in ATP production, which powers ion pumps (e.g., sodium-potassium ATPase), critical for maintaining ion gradients across the cell membrane.
Phosphate: Participates in energy metabolism and serves as a buffer, maintaining acid-base balance during ischemia and reperfusion.

Balancing these electrolytes could stabilize the heart rhythm, especially during post-arrest conditions where arrhythmias and electrolyte imbalances often occur. For instance, correcting potassium levels may prevent arrhythmias, while optimizing calcium can improve contractility, leading to more efficient cardiac output.

### Technical Integration:

Developing a formulation that integrates these electrolytes with miRNA therapies aims to stabilize heart function during and after cardiac arrest. Nanoparticle delivery systems ensure targeted, sustained release of electrolytes and miRNAs into the heart, enhancing therapeutic efficacy.
Aim 3: Evaluation of Human Plasma-Derived Antithrombin in Coagulopathy Management Mechanism of Antithrombin:

Antithrombin is a natural anticoagulant that inactivates thrombin and other proteases involved in blood clotting (e.g., factor Xa). In the context of cardiac arrest, where coagulopathy (excessive clotting) can lead to complications such as thromboembolism, human plasma-derived antithrombin can mitigate clot formation.
Thrombin Inhibition: Antithrombin binds to and inactivates thrombin, a key enzyme in the conversion of fibrinogen to fibrin during clot formation.
Synergy with Heparin: Antithrombin's activity is enhanced by heparin, a naturally occurring glycosaminoglycan, increasing its anticoagulant properties. This prevents excessive clot formation during and after resuscitation from cardiac arrest, reducing the risk of embolic events. By incorporating human plasma-derived antithrombin into this therapy, the aim is to restore hemostasis while preventing thrombotic complications during recovery from cardiac arrest.

### Technical Process:

The proposed antithrombin extraction method from human plasma involves apheresis, followed by purification via affinity chromatography. Antithrombin is concentrated and dialyzed post-purification, with quality control steps to ensure the protein's purity and activity.

### Conclusion and Mechanistic Integration

The multifaceted approach described in this research integrates three core mechanisms: miRNA-based gene regulation to prevent fibrosis, apoptosis, and inflammation in cardiac tissue post-arrest.

Electrolyte stabilization to maintain proper cardiac electrophysiology and cellular homeostasis, minimizing arrhythmias and supporting heart muscle function.

Antithrombin administration to prevent coagulation-related complications, ensuring the heart and vasculature remain stable during recovery.

These mechanisms address multiple aspects of cardiac arrest pathophysiology, offering a novel combination therapy (CARDIOGARDIA) aimed at improving patient outcomes through a precision medicine approach. The integration of miRNA regulation, electrolyte balance, and anticoagulation could theoretically lead to more effective interventions in cardiac arrest, bridging the gap between basic molecular research and clinical applications.

### REFERENCES

1—Karthik Gonuguntla, Muchi Ditah Chobufo, Shaik A, et al. Trends in cardiac arrest mortality in the US, 1999-2020. medRxiv (Cold Spring Harbor Laboratory). Published online August 2, 2023. doi:https://doi.org/10.1101/2023.08.01.23293526
2—Empana JP, Lerner I, Valentin E, et al. Incidence of Sudden Cardiac Death in the European Union. Journal of the American College of Cardiology. 2022; 79(18):1818-1827. doi:https://doi.org/10.1016/j.jacc.2022.02.041
3—Reinier K, Dizon B, Chugh H, et al. Warning symptoms associated with imminent sudden cardiac arrest: a population-based case-control study with external validation. The Lancet Digital Health. 2023; 5(11). doi:https://doi.org/10.1016/s2589-7500(23)00147-4
4—1.Feinberg MW, Moore KJ. MicroRNA Regulation of Atherosclerosis. Circulation Research. 2016; 118(4):703-720. doi:https://doi.org/10.1161/circresaha.115.306300
5—1.Rodgers GM, Arash Mahajerin. Antithrombin Therapy: Current State and Future Outlook. Clinical and applied thrombosis/hemostasis. 2023; 29. doi:https://doi.org/10.1177/10760296231205279
6—1.Jaki T, Burdon A, Chen X, Pavel Mozgunov, Zheng H, Baird R. Early phase clinical trials in oncology: Realising the potential of seamless designs. European journal of cancer. 2023; 189:112916-112916. doi:https://doi.org/10.1016/j.ejca.2023.05.005
7—1.Kong YW, Baqar S, Jerums G, Ekinci EI. Sodium and Its Role in Cardiovascular Disease—The Debate Continues. Frontiers in Endocrinology. 2016; 7(164). doi:https://doi.org/10.3389/fendo.2016.00164
8—Hazra A, Gogtay N. Biostatistics series module 3: Comparing groups: Numerical variables. Indian Journal of Dermatology. 2016; 61(3):251. doi:https://doi.org/10.4103/0019-5154.182416
9—1. Yu S, Wu C, Zhu Y, Diao M, Hu W. Rat model of asphyxia-induced cardiac arrest and resuscitation. Frontiers in Neuroscience. 2023; 16:1087725. doi:https://doi.org/10.3389/fnins.2022.1087725
10—https://www.mpi-hlr.de/en
11—https://www.mpg.de/149809/heart-lung-research
12—1.Sridharan D, Pracha N, Rana SJ, et al. Preclinical Large Animal Porcine Models for Cardiac Regeneration and Its Clinical Translation: Role of hiPSC-Derived Cardiomyocytes. Cells. 2023; 12(7):1090. doi:https://doi.org/10.3390/cells12071090
13—ROSKOVA I, VECERA M, LENKA RADOVA, et al. Small RNA Sequencing Identifies a Six-MicroRNA Signature Enabling Classification of Brain Metastases According to their Origin. Cancer Genomics & Proteomics. 2022; 20(1):18-29. doi:https://doi.org/10.21873/cgp.20361
14—Zeng M, Wei X, Zhou J, Luo S. LncRNA PART1 Attenuates Myocardial Ischemia-Reperfusion Injury by Regulating TFAP2C/DUSP5 Axis via miR-302a-3p. Korean Circulation Journal. 2024; 54(5):233-233. doi:https://doi.org/10.4070/kcj.2023.0131
15—Lee S. Cardiovascular Disease and miRNAs: Possible Oxidative Stress-Regulating Roles of miRNAs. Antioxidants. 2024; 13(6):656-656. doi:https://doi.org/10.3390/antiox13060656
16—miRNA Mimics & Inhibitors|Thermo Fisher Scientific—US. Thermofisher.com. Published 2024. Accessed October 16, 2024. https://www.thermofisher.com/us/en/home/life-science/epigenetics-noncoding-rna-research/mirna-analysis/mirna-mimics-inhibitors.html
17—Sigma Aldrich. NMR Chemical Shifts of Impurities Charts. Merck. 2024; 1(1). https://www.sigmaaldrich.com/MX/en/technical-documents/technical-article/genomics/cloning-and-expression/blue-white-screening
18—miRNA Mimics & Inhibitors|Thermo Fisher Scientific—US. Thermofisher.com. Published 2024. Accessed October 16, 2024. https://www.thermofisher.com/us/en/home/life-science/epigenetics-noncoding-rna-research/mirna-analysis/mirna-mimics-inhibitors.html
19-Rajagopal AS, Champney TH. Teaching Without Harm: The Ethics of Performing Posthumous Procedures on the Newly Deceased. Cureus. Published online December 2, 2020. doi:https://doi.org/10.7759/cureus.11855
20—Choi Y, Kim A, Kim J, Lee J, Lee SY, Kim C. Optimization of RNA Extraction from Formalin-Fixed Paraffin-Embedded Blocks for Targeted Next-Generation Sequencing. Journal of Breast Cancer. 2017; 20(4):393. doi:https://doi.org/10.4048/jbc.2017.20.4.393
21—Sampaio-Silva F, Magalhães T, Carvalho F, Dinis-Oliveira RJ, Silvestre R. Profiling of RNA Degradation for Estimation of Post Morterm Interval. Kayser M, ed. PLoS ONE. 2013; 8(2):e56507. doi:https://doi.org/10.1371/journal.pone.0056507
22—Kramer MF. Stem-Loop RT-qPCR for miRNAs. Current Protocols in Molecular Biology. 2011; 95(1). doi:https://doi.org/10.1002/0471142727.mb1510s95
23—de Gonzalo-Calvo D, Marchese M, Hellemans J, et al. Consensus guidelines for the validation of qRT-PCR assays in clinical research by the CardioRNA consortium. Molecular Therapy-Methods & Clinical Development. 2022; 24:171-180. doi:https://doi.org/10.1016/j.omtm.2021.12.007
24—Kura B, Szeiffova Bacova B, Kalocayova B, Sykora M, Slezak J. Oxidative Stress-Responsive MicroRNAs in Heart Injury. International Journal of Molecular Sciences. 2020; 21(1):358. doi:https://doi.org/10.3390/ijms21010358
25—Takahashi M, Ogura K, Goto T, Hayakawa M. Electrocardiogram monitoring as a predictor of neurological and survival outcomes in patients with out-of-hospital cardiac arrest: a single-center retrospective observational study. Frontiers in Neurology. 2023; 14. doi:https://doi.org/10.3389/fneur.2023.1210491
26—Sessa F, Salerno M, Esposito M, Cocimano G, Pomara C. miRNA Dysregulation in Cardiovascular Diseases: Current Opinion and Future Perspectives. International Journal of Molecular Sciences. 2023; 24(6):5192. doi:https://doi.org/10.3390/ijms24065192

1. (Composition Claim):
A pharmaceutical composition for treating cardiac arrest, comprising:
microRNAs chosen from the group consisting of miR-21, miR-1, miR-29, miR-17, and miR-133.
antithrombin; and
electrolytes;
in which the composition is admixed with a pharmaceutically acceptable carrier.

2. (Method of Use Claim):
A method for treating cardiac arrest in a subject in need thereof, comprising administering to the subject a therapeutically effective amount of the composition of claim 1, whereby one or more pathological processes selected from the group consisting of fibrosis, apoptosis, arrhythmia, and coagulopathy are diminished.
